(19) United States
(12) Patent Application Publication
(10) Pub. No.: US 2024/0366203 A1
(43) Pub. Date: Nov. 7, 2024

(54) MEDICAL DEVICES AND RELATED METHODS FOR DELIVERING A MATERIAL

(71) Applicants: Boston Scientific Scimed, Inc., Maple Grove, MN (US); Boston Scientific Medical Device Limited, Galway (IE)

(72) Inventors: Deepak Kumar SHARMA, Muzaffarnagar (IN); Sharath Kumar G, Kanakapura (IN); Kevin L. BAGLEY, Natick, MA (US); Mazen YATIM, Southborough, MA (US); Samuel GOSHEN, Brookline, MA (US); Rajivkumar SINGH, Thane (IN); Nachiket GOLE, Pune (IN)

(73) Assignees: Boston Scientific Scimed, Inc., Maple Grove, MN (US); Boston Scientific Medical Device Limited, Galway (IE)

(21) Appl. No.: 18/650,186

(22) Filed: Apr. 30, 2024

Related U.S. Application Data
(60) Provisional application No. 63/499,553, filed on May 2, 2023.

(51) Int. Cl. A61B 17/00; A61B 90/00

(52) U.S. Cl. CPC A61B 17/00491; A61B 17/00234; A61B 90/08; A61B 2017/00296; A61B 2017/00367; A61B 2017/0046; A61B 2017/00477; A61B 2017/00495; A61B 2017/00544; A61B 2017/00893; A61B 2090/0807

(57) ABSTRACT

A medical device may comprise a handle having a fluid source, a conduit coupled to the handle, and a distal portion coupled to a distal end of the conduit. The distal portion may comprise an outer portion defining a lumen and an inner portion. The inner portion may be disposed within the lumen if the outer portion. The inner portion may define a cavity having a distal opening. The cavity may be configured to contain a material. In a first configuration of the distal portion, the material is disposed within the inner portion. Delivery of a fluid from the fluid source into the lumen of the outer portion transitions the distal portion from the first configuration to a second configuration and causes the inner portion to move distally relative to the outer portion and deliver at least a portion of the material.

### CROSS-REFERENCE TO RELATED APPLICATIONS

This application claims the benefit of priority of U.S. Provisional Patent Application No. 63/499,553, filed on May 2, 2023, the entirety of which is incorporated herein by reference.

### TECHNICAL FIELD

This disclosure generally relates to medical devices and related methods that may be used to treat a subject. More particularly, at least some embodiments of the present disclosure relate to devices and related methods for delivering a material.

### BACKGROUND

In certain medical procedures, it may be necessary to remove a portion of a tissue and/or to stop or minimize bleeding internal to the body. For example, some endoscopic medical procedures may require removing tissue (e.g., polyps, lesions, etc.) within the gastrointestinal tract, for example, in the esophagus, stomach, or intestines. Other procedures may require hemostasis of bleeding tissue within the gastrointestinal tract.

Physicians have adopted minimally invasive techniques for removing tissue, such as endoscopic procedures like endoscopic mucosal resection (EMR) and endoscopic submucosal dissection (ESD). EMR methods are typically used for removal of small cancerous or abnormal tissues (e.g., polyps), and ESD methods are typically used for en bloc removal of large cancerous or abnormal tissues (e.g., lesions). Physicians have also adopted minimally invasive techniques to stop bleeding internal to the body.

In these procedures, a material (e.g., fluid, substance, powder, and/or agent) may be delivered to a treatment site within the gastrointestinal tract using systems or devices. Such systems or devices, however, may require medical professionals to exert or apply great amounts of force to deliver the material to the treatment site. Accordingly, medical professionals may experience fatigue and/or discomfort from exerting large amounts of force over the course of a procedure. Over time, for example, medical professionals may experience symptoms similar to those of Carpal Tunnel Syndrome, tendonitis, or De Quervain's tenosynovitis. When a medical professional experiences fatigue or other pain in the fingers, hand, or wrist, the medical professional may take breaks from the procedure and/or readjust his or her hand position. When a medical professional takes breaks and/or repeatedly readjusts his or her handgrip in between procedure tasks, the procedure may be prolonged and procedural tasks may be more difficult.

Furthermore, the amounts of forces may pose a risk of various components of the device breaking over the course of the procedure. If the devices break, for example, within a patient, the patient may have an increased risk of harm or injury. For example, the broken devices may perforate, cut, puncture, or otherwise injure the patient's gastrointestinal tract.

The devices and methods of this disclosure may rectify some of the deficiencies described above or address other aspects of the art.

### SUMMARY

This disclosure includes medical devices and related methods for delivering a material. Each of the aspects disclosed herein may include one or more features described in connection with any of the other disclosed aspects.

In aspects, a medical device may include a handle having a fluid source, a conduit coupled to the handle, and a distal portion coupled to a distal end of the conduit. The distal portion may include an outer portion defining a lumen, and an inner portion. The inner portion may define a cavity having a distal opening. The cavity may be configured to contain a material. In a first configuration of the distal portion, the material may be disposed within the inner portion and the inner portion is disposed within the lumen of the outer portion. Delivery of a fluid from the fluid source into the lumen of the outer portion may transition the distal portion from the first configuration to a second configuration and cause the inner portion to move distally relative to the outer portion. At least a portion of the material may be delivered.

Any of the aspects disclosed herein may include any of the following features in any combination. The fluid may be a gas. The handle may include a first arm and a second arm. The fluid source may be a sac disposed between the first arm and the second arm. Upon movement of the first arm towards the second arm, the sac may be compressed. The sac may be corrugated. The distal portion may be detachable from the conduit.

In the first configuration of the medical device, the inner portion may have a first length. In the second configuration, the inner portion may have a second length. The first length may be greater than the first length. The conduit may be flexible, and the distal portion of the medical device may be rigid or semi-rigid.

The inner portion may be a bladder. A color of the bladder may indicate a volume of the bladder. The outer portion may include a distal valve through which the material may be delivered. The valve may be a one-way valve. The inner portion and the outer portion may form one continuous structure. In the first configuration, the inner portion may be disposed within the outer portion. In the second configuration, the inner portion may extend distally relative to the outer portion. In the second configuration, the inner portion may be turned inside out to release the material. The material may be a patch.

In another aspect, a distal end portion of a medical device may include an outer portion defining a lumen and an inner portion defining a cavity configured to contain the material. Delivery of a fluid into the lumen of the outer portion may transition the distal portion from a first configuration to a second configuration and cause the inner portion to move distally relative to the outer portion. In the second configuration, the inner portion may be turned inside out to release at least a portion of the material.

Any of the aspects disclosed herein may include any of the following features in any combination. The material may be a patch. The inner portion and the outer portion may form one continuous structure. The distal end portion may further include a fitting on a proximal end of the distal end portion. The fitting may be configured to removably couple the distal end portion to a conduit.

In an aspect, a medical method may include inserting a distal end portion of a medical device into a subject. The distal end portion may include an outer portion defining a lumen and an inner portion defining a cavity configured to contain a material. During insertion of the distal end portion of the medical device into the subject, the inner portion may be disposed within the lumen of the outer portion. The method may further include delivering a fluid to the lumen of the outer portion. Upon delivery of the fluid to the lumen of the outer portion, the inner portion may move distally relative to the outer portion to deliver at least a portion of the material.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 is a perspective view of a medical device, according to aspects of this disclosure.

FIGS. 2A-2B are cross-sectional schematic views of a distal portion of the medical device of FIG. 1 in a first configuration (FIG. 2A) and a second configuration (FIG. 2B), according to aspects of this disclosure.

FIGS. 2C-2D are cross-sectional views of a distal end of the medical device of FIG. 1 in the first configuration (FIG. 2C) and the second configuration (FIG. 2D), according to aspects of this disclosure.

FIG. 3 is a flow diagram of an exemplary method, according to aspects of this disclosure.

FIGS. 4A-4B are cross-sectional views of an alternative distal portion of the medical device of FIG. 1 in a first configuration (FIG. 4A) and a second configuration (FIG. 4B), according to aspects of this disclosure.

FIG. 5 is a perspective view of an alternative handle of the medical device of FIG. 1, according to aspects of this disclosure.

FIG. 6 is a side view of a further alternative handle of the medical device of FIG. 1, according to aspects of this disclosure.

FIG. 7 is a perspective view of an alternative handle of the medical device of FIG. 1, according to aspects of this disclosure.

### DETAILED DESCRIPTION

Reference will now be made in detail to aspects of the present disclosure, examples of which are illustrated in the accompanying drawings. Wherever possible, the same or similar reference numbers will be used through the drawings to refer to the same or like parts. The term “distal” refers to a portion farthest away from a user when introducing a device into a subject (e.g., patient). By contrast, the term “proximal” refers to a portion closest to the user when placing the device into the subject. Proximal and distal directions are labeled with arrows marked “P” and “D”, respectively, throughout various figures.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

Although the treatment site is discussed herein as being in the subject's GI tract, this disclosure is not so limited, as the treatment site may be any internal lumen, organ, cavity, or other tissue within the subject. Additionally, although endoscopes are referenced herein, it will be appreciated that the disclosure encompasses any medical devices having a working channel extending from a proximal end to a distal end, such as ureteroscopes, duodenoscopes, gastroscopes, endoscopic ultrasonography (“EUS”) scopes, colonoscopes, bronchoscopes, laparoscopes, arthroscopes, cystoscopes, aspiration scopes, sheaths, or catheters.

Embodiments of the disclosure may address one or more of the limitations in the art. The scope of the disclosure, however, is defined by the attached claims and not the ability to solve a specific problem.

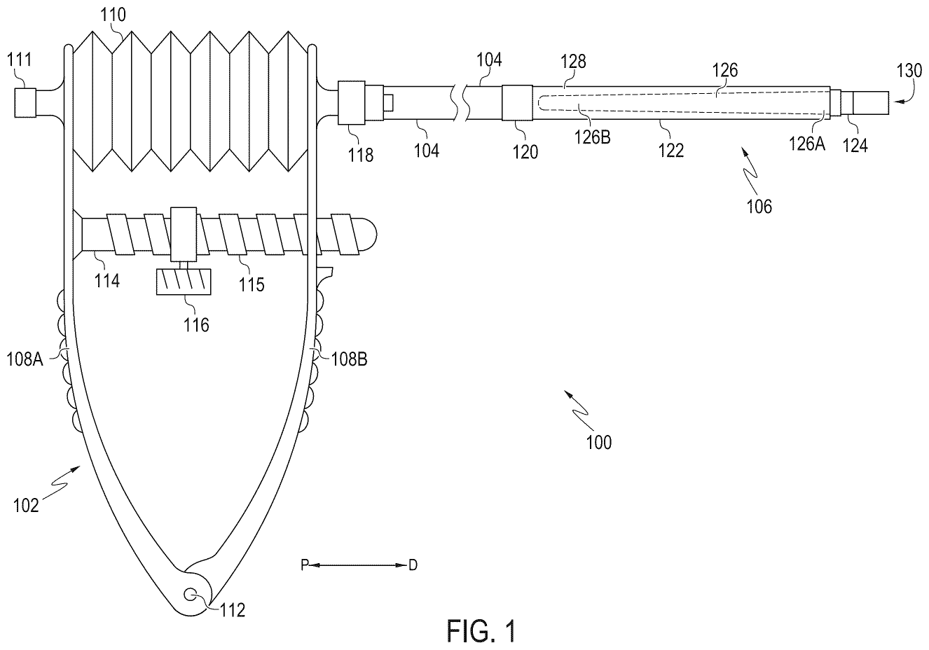

FIG. 1 illustrates a medical device 100 having a handle 102, a conduit 104, and a distal portion 106. In some embodiments, handle 102, conduit 104, and/or distal portion 106 may be formed as one device with these parts permanently fixed to one another. In alternative embodiments, one or more of handle 102, conduit 104, and/or distal portion 106 may be attachable or detachable from the other parts. For example, conduit 104 may be configured to couple/decouple from handle 102. Additionally or alternatively, for example, distal portion 106 may be configured to couple/decouple from conduit 104. In such a way, handle 102, conduit 104, and/or distal portion 106 may be reusable and/or single-use. Handle 102, conduit 104, and distal portion 106 may each be comprised of one or more biocompatible materials.

Handle 102 may be configured similar to a bellows. For example, handle 102 may include a first arm 108A, a second arm 108B, and a sac 110. Sac 110 may be disposed between a first end of first arm 108A and a first end of second arm 108B. For example, as first arm 108A is moved towards second arm 108B, and/or vice versa, sac 110 may be compressed. As first arm 108B is moved away from second arm 108B, and/or vice versa, sac 110 may be expanded.

Sac 110 may include a proximal valve 111 (e.g., a one-way valve or a check valve) configured to enable fluid flow into sac 110, for example, as sac 110 is expanded, and prevent fluid flow proximally out of sac 110, for example, as sac 110 is compressed. In some embodiments, sac 110 may be corrugated, for example, to facilitate the expansion and compression of sac 110. Conduit 104 may be fluidly coupled to sac 110 such that, as sac 110 is compressed, a fluid contained within sac 110 is forced into a lumen (not shown) of conduit 104. The fluid within sac 110 may be a fluid such as a gas (e.g., air, oxygen, nitrogen, etc.) or a liquid (e.g., water, saline, agents, etc.). Accordingly, although not shown, proximal valve 111 may be coupled to a fluid source (e.g., a compressor, a pump, etc.) in some embodiments. The lumen (not shown) of conduit 104 may extend along a longitudinal length of conduit 104, for example, from a proximal end to a distal end of conduit 104. In such a way, fluid may flow between handle 102 (e.g., sac 110) and distal portion 106 via the lumen of conduit 104.

In some embodiments, handle 102 may include an indicator (not shown). In some embodiments, the indicator may be coupled to sac 110. The indicator may be, for example, a gauge, an instrument, a device, etc. configured to measure one or more aspects of medical device 100. For example, the indicator (not shown) may be configured to measure and/or display a volume, a pressure, a temperature, etc. of one or more portions of medical device 100 (e.g., sac 110 and/or distal portion 106). Additionally or alternatively, the indicator may be configured to measure and/or display a volume of material delivered via distal portion 106. The indicator may, for example, include an analog display and/or a digital display.

A second end of first arm 108A and a second end of second arm 108B may be movably coupled via a joint 112. Joint 112 may be, for example, a hinge joint (e.g., a mechanical hinge, a living hinge, etc.), a saddle joint, a ball and socket joint, a pivot joint, etc. In such a way, first arm 108A may pivot about joint 112 relative to second arm 108B and/or vice versa. As first arm 108A pivots about joint 112 towards second arm 108B and/or vice versa, sac 110 is compressed between first arm 108A and second arm 108B. First arm 108A and/or second arm 108B may include one or more features (e.g., grooves, indentations, raised surfaces, etc.) to facilitate a user's grip on handle 102.

Additionally or alternatively, in some embodiments, the first end of first arm 108A and/or the first end of second arm 108B may translate along a mechanism (e.g., a bar, a rail, a tube, etc.) extending between each of the first ends. For example, the mechanism may be contained partially or completely within sac 110 and hidden in FIG. 1. In some embodiments, the mechanism may assist in movement of first arm 108A and/or second arm 108B. For example, the mechanism may assist in aligning first arm 108A relative to second arm 108B, or vice versa. Additionally or alternatively, the mechanism may limit the movement of first arm 108A relative to second arm 108B. In such a way, the compression of sac 110 may be limited.

In some embodiments, a rod 114 may extend between first arm 108A and second arm 108B, for example, from a surface of first arm 108A through a channel (not shown) on second arm 108B. In some embodiments, rod 114 may include a plurality of ridges 115 (e.g., teeth). Although not shown, plurality of ridges 115 may be configured to engage with one or more features (e.g., a pawl, a cutout, etc.) within the channel on second arm 108B, for example, similar to a cable tie or a zip tie. In such a way, first arm 108A may be locked in a position relative to second arm 108B, or vice versa.

Additionally or alternatively, a movable lock 116 may be disposed along rod 114, for example, between first arm 108A and second arm 108B. In some embodiments, movable lock 116 may be configured to limit movement of first arm 108A relative to second arm 108B, or vice versa. As described above, movement of first arm 108A relative to second arm 108B may compress or expand sac 110. Accordingly, movable lock 116 may be disposed along a length of rod 114 to limit the movement of first arm 108A relative to second arm 108B. In such a way, the compression of sac 110 may be limited. Movable lock 116 may be relocated along a length of rod 114, for example, to increase or decrease the amount of compression of sac 110.

Additionally or alternatively, movable lock 116 may be disposed along rod 114, for example, such that second arm 108B is disposed between first arm 108A and movable lock 116. In such an example, movable lock 116 may be configured, for example, to prevent movement of second arm 108B past movable lock 116. In such a way, movable lock 116 may limit the expansion of sac 110. Movable lock 116 may be relocated on rod 114, for example, to increase or decrease the expansion of sac 110.

In further alternative embodiments, two or more movable locks 116 may be used, for example, to limit the compression and expansion of sac 110. For example, one movable lock 116 may be positioned on rod 114 between first arm 108A and second arm 108B and another movable lock 116 may be positioned on rod 114 such that second arm 108B is between the two movable locks 116.

Still referring to FIG. 1, conduit 104 may be coupled to a distal end of handle 102. Conduit 104 may be formed by a tube, a sheath, a cannula, etc. Conduit 104 may be flexible and configured to move through tortuous pathways of body lumens of a patient. In some embodiments, conduit 104 may be coupled or uncoupled from handle 102 via, for example, a first fitting 118 (e.g., a luer lock or other removable fitting commonly known in the art). Alternatively, conduit 104 may be fixedly coupled to handle 102.

One or more dimensions of conduit 104 may vary. For example, a length of conduit 104 may be approximately about 5-178 inches long, for example, between 25-100 inches. Conduit 104 may be longer or shorter. A cross-section of conduit 104 may be circular, ovular, rectangular, square, or any other shape commonly known in the art. Conduit 104 may have an outer diameter of approximately about 0.03-0.80 inches, for example, between 0.04-0.50 inches. Additionally, an inner diameter of conduit 104 may be approximately about 0.01-0.79 inches, for example, between 0.01-0.49 inches. Conduit 104 may have a larger or smaller outer diameter and/or inner diameter.

Although not shown, conduit 104 may include additional lumens (e.g., a second lumen, a third lumen, etc.). The additional lumens of conduit 104 may each be fluidly coupled to sac 110. Accordingly, as fluid is forced from sac 110 through conduit 104, fluid flows through each lumen fluidly coupled to sac 110. Additionally or alternatively, a proximal end of the additional lumens of conduit 104 may include a port (not shown). In such a way, the additional lumens may be configured to receive one or more accessory medical devices (e.g., baskets, forceps, scopes, etc.) and/or one or more materials.

Distal portion 106 may be fixedly or removable coupled to a distal end of conduit 104. For example, distal portion 106 may be fixedly coupled to the distal end of conduit 104. In alternative embodiments, distal end of conduit 104 may include a second fitting 120 (e.g., a luer lock or other removable fitting commonly known in the art). In such an embodiment, distal portion 106 may be coupled/uncoupled from conduit 104.

Distal portion 106 may include a receptacle 122, a distal valve 124, and a bladder 126 disposed within receptacle 122. Receptacle 122 may, in some embodiments, be formed similar to a barrel or a tube, such that, for example, a lumen 128 is defined within receptacle 122. Lumen 128 may be in fluid communication with the lumen of conduit 104, for example, to enable or permit fluid flow from conduit 104 into receptacle 122.

Receptacle 122 may be rigid or semi-rigid. For example, receptacle 122 may be comprised of a plastic, a metal, a polymer, a rubber, or any other material commonly used in the art. Accordingly, in some embodiments, receptacle 122 may be flexible and configured to move through tortuous pathways of body lumens of a patient. In some embodiments, receptacle 122 may be transparent or semi-transparent, for example, to permit visualization of bladder 126 disposed within receptacle 122. Additionally or alternatively, receptacle 122 may be colored (e.g., blue, green, red, black, etc.), for example, to visually indicate one or more characteristics of distal portion 106 (e.g., a volume of bladder 126, a length of receptacle 122, etc.). For example, a blue receptacle 122 may indicate a first volume of bladder 126 and/or a first length of receptacle 122, and a green receptacle 122 may indicate a second volume of bladder and/or a second length of receptacle 122, etc., so that a user may select a volume of agent/fluid desired for delivery.

Receptacle 122 may have a longitudinal length of approximately 0.10-5.00 inches, for example, between 0.50-2.00 inches. Accordingly, receptacle 122 may be configured to contain approximately 0.01-25 milliliters of material (e.g., 0.01-24 milligrams). Receptacle 122 may be similarly sized and/or shaped to conduit 104. For example, a cross-section of receptacle 122 may be circular, ovular, rectangular, square, or any other shape commonly known in the art. Receptacle 122 may have a same cross-sectional shape as conduit 104 or a different cross-sectional shape. For example, conduit 104 may be circular, and receptacle 122 may be square, or vice versa.

Receptacle 122 may have an outer diameter of approximately 0.03-0.80 inches, for example, between 0.04-0.50 inches. The outer diameter of receptacle 122 may be the same or similar to the outer diameter of conduit 104. Alternatively, the outer diameter of receptacle 122 may be larger or smaller than the outer diameter of conduit 104. Additionally, an inner diameter of receptacle 122 may be approximately 0.01-0.79 inches, for example, between 0.01-0.49 inches. The inner diameter of receptacle 122 may be the same or similar to the inner diameter of conduit 104. Alternatively, the inner diameter of receptacle 122 may be larger or smaller than the inner diameter of conduit 104.

Bladder 126 may be disposed within receptacle 122, for example, within lumen 128. In such a way, bladder 126 extends proximally within receptacle 122. Bladder 126 may be positioned within receptacle 122 such that the opening of distal portion 126A of bladder 126 is in a distal-facing direction. For example, distal portion 126A of bladder 126 may be coupled to the distal end of receptacle 122 and/or to a proximal end of distal valve 124, for example, between receptacle 122 and distal valve 124. A proximal end 126B of bladder 126 may be movable within receptacle 122. For example, proximal end 126B of bladder 126 may be detached from receptacle 122. Bladder 126 may extend proximally along an entire longitudinal length of receptacle 122. Alternatively, bladder 126 may partially extend along a longitudinal length of receptacle 122.

Bladder 126 may be comprised of one or more flexible materials. For example, bladder 126 may be comprised of rubber, latex, nylon, polyurethane, Pebax®, silicone, etc. Bladder 126 may, for example, be comprised of materials similarly used for medical balloons. Bladder 126 may be colored, transparent, and/or semi-transparent. For example, bladder 126 may be colored to visually indicate a volume of bladder 126 and/or characteristics of the material disposed within bladder 126, so that a user may select a volume or type of agent/fluid desired for delivery.

Distal valve 124 may be fixedly coupled to a distal end of receptacle 122. Distal valve 124 may have a distalmost opening 130. Distal valve 124 may be configured as a one-way valve (e.g., a duckbill valve, a check valve, etc.), for example, to permit fluid flow in a distal direction (e.g., out of distal portion 106) and prevent fluid flow in a proximal direction (e.g., into distal portion 106).

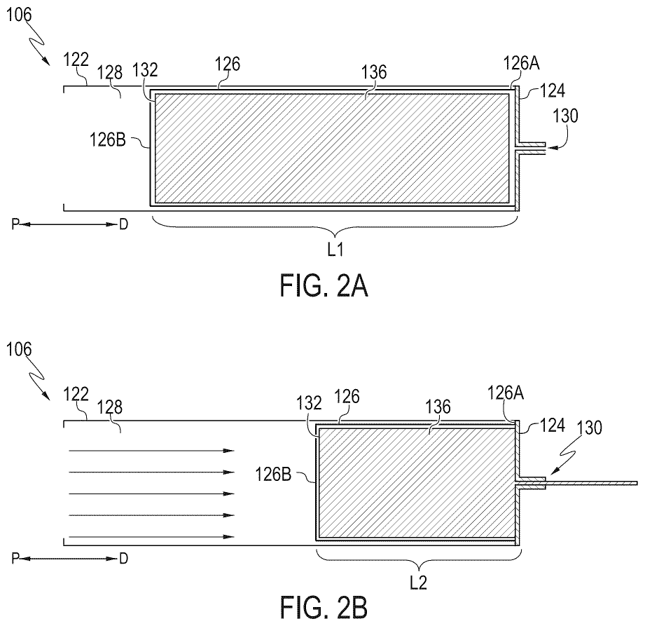

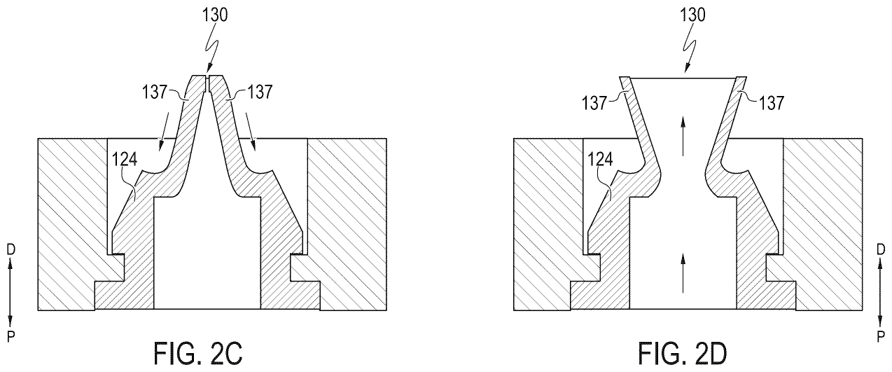

FIGS. 2A-2B are cross-sectional illustrations of distal portion 106 shown in FIG. 1 in a first configuration (FIG. 2A) and a second configuration (FIG. 2B). With the opening of bladder 126 facing a distal direction, a cavity 132 is formed within bladder 126. As shown in FIGS. 2A-2B, a material 136 (e.g., a gel, a liquid, a powder, a patch, an agent, etc.) may be disposed within cavity 132. Bladder 126 may be complementarily sized and/or shaped according to receptacle 122. For example, a cross-sectional shape of bladder 126 and the cross-sectional shape of receptacle 122 may be circular. Furthermore, an outer diameter of bladder 126 may be the same or smaller than an inner diameter of receptacle 122.

As shown in FIG. 2A, bladder 126 may extend proximally within receptacle 122 by a first length (“L1”) in the first configuration. Material 136 may be disposed within cavity 132 of bladder 126 in this configuration. Additionally, as shown in FIG. 2C, distal valve 124 is closed in the first configuration. For example, a pair of lips 137 forming distal opening 130 of distal valve 124 may touch or abut, thus providing a seal. In such a way, lips 137 may assist in preventing external fluids (e.g., bodily fluids) from flowing proximally into cavity 132 of bladder 126.

In the second configuration, material 136 may be delivered through distal valve 124. To deliver material 136 from cavity 132, a fluid is injected into receptacle 122 via conduit 104. The fluid may be injected by, for example, compressing sac 110, as described above with reference to FIG. 1. The injected fluid fills lumen 128 of receptacle 122 such that, as receptacle 122 fills with the injected fluid, bladder 126 is forced distally within receptacle 122. Accordingly, a longitudinal length of bladder 126 may decrease such that bladder 126 has a smaller, second length (“L2”) in the second configuration. In other words, first length L1 may be greater than second length L2. Furthermore, as bladder 126 is forced distally within receptacle 122, the volume of cavity 132 thus decreases. Accordingly, material 136 is forced distally through distal valve 124. For example, as shown in FIG. 2D, as a distal force is exerted on lips 137 by material 136, each of lips 137 is forced outward. With lips 137 forced outward, distal valve 124 is opened, and material 136 is delivered to a target tissue.

A variety of materials may be delivered with medical device 100 (e.g., with distal portion 106). Material 136 may include any compound or agent used for a diagnosis, a treatment, and/or palliation of a patient's disease or injury. For example, material 136 may include one or more hemostatic material(s) or agent(s). As previously described, material 136 may be a liquid, a gel, and/or a powder.

Additionally or alternatively, material 136 may be a first part of a multi-part material (e.g., a two-, three-, or four-part material). For example, once material 136 is combined with a second material, the combined material may undergo a reaction (e.g., coagulation or other chemical reaction). The additional part(s) of the multi-part material may be delivered via a working channel of an endoscope, an auxiliary medical device, a second of medical device 100, etc. In some embodiments, distal portion 106 may include one or more ports (not shown), for example, on an external surface of receptacle 122. The port(s) may be configured to deliver a second material, for example, to activate the reaction of material 136 once material 136 has been delivered.

In some embodiments, the port(s) may be configured to deliver the fluid injected into receptacle 122 to the treatment site. In such a way, material 136 and the second material (e.g., the fluid injected into receptacle 122) are combined immediately or shortly after material 136 has been delivered to the treatment site. The port(s) may be configured to permit fluid flow in a first direction (e.g., out of receptacle 122) and prevent fluid flow in a second direction (e.g., into receptacle 122). In some embodiments, the one or more ports (not shown) may be angled such that the fluid is delivered distally relative to the port(s). For example, the port(s) may be oriented at an angle between 0-80 degrees away from the external surface of receptacle 122 such that the ports deliver the fluid distally relative to distal portion 106.

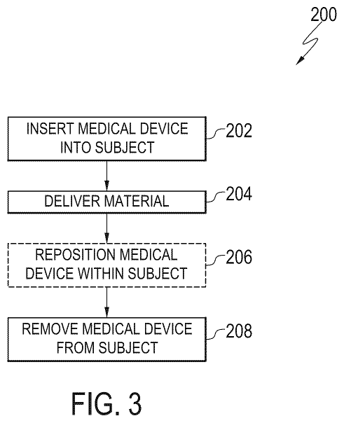

FIG. 3 is a flow diagram of an exemplary method 200 that one or more users may perform with any of the medical devices discussed herein, for example distal portion 106 of medical device 100. Medical device 100 may include handle 102 and distal portion 106. In a first step 202 of method 200, medical device 100 (e.g., distal portion 106) may be inserted into the subject, for example, though a natural orifice or an incision. In some examples, medical device 100 may be inserted into the patient using an endoscope (not shown), for example, via a working channel of the endoscope. First step 202 may further include positioning distal portion 106 of medical device 100 and/or deflecting and/or positioning the endoscope (not shown) via knobs, levers, dials, etc.

Once distal portion 106 is in the desired position, a second step 204 includes delivering at least part of a material 136 contained within receptacle 122 of distal portion 106, for example, within bladder 126. Material 136 may be delivered, for example, by manipulating one or more portions of handle 102. For example, first arm 108A and/or second arm 108B may be moved such that sac 110 forces a fluid through conduit 104 and into receptacle 122, as discussed above. As the fluid is injected into receptacle 122, bladder 126 is forced distally within receptacle 122 and the material contained within cavity 132 of bladder 126 is delivered, for example, through distal valve 124.

In an optional step 206 of method 200, for example, before, during, or after at least a portion of material 136 is delivered, the user(s) may reposition distal portion 106 within the subject. For example, the user(s) may move medical device 100 proximally or distally within the subject for a subsequent delivery of material 136 to another site. Additionally or alternatively, the user(s) may move endoscope proximally or distally within the subject and/or deflect the distal portion of the endoscope via knobs, levers, dials, etc.

Once the desired volume of material 136 is delivered, the user(s) may remove medical device 100 from the subject, in a step 208. For example, medical device 100 may be removed by pulling medical device 100 and/or the endoscope proximally through the natural orifice or incision. Once medical device 100 is removed, method 200 may be repeated. For example, the user(s) may use a second medical device 100. The second medical device 100 may include the same material 136 as the first medical device 100, or a different material. Additionally or alternatively, the user(s) may detach or uncouple the used distal portion 106 from medical device 100 and attach or couple a second, unused distal portion 106 to medical device 100. The second distal portion 106 may include the same material 136 as the first distal portion 106 or a different material. Method 200 may be repeated as desired, for example to deliver the desired volume of material 136.

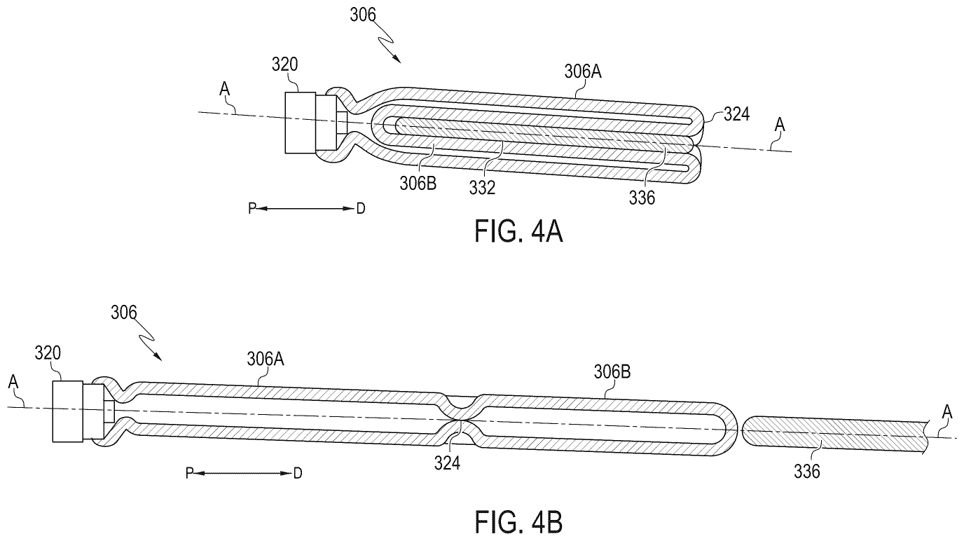

FIGS. 4A and 4B are cross-sectional views of an alternative distal portion 306 in a first configuration (FIG. 4A) and in a second configuration (FIG. 4B). Distal portion 306 may be used with handle 102 and/or conduit 104, discussed above with respect to FIG. 1. For example, in some embodiments, distal portion 306 may include a fitting 320. Fitting 320 may be configured to couple/uncouple distal portion 306 from conduit 104 of FIG. 1.

Distal portion 306 may include a first portion 306A and a second portion 306B. In the first configuration, second portion 306B may be disposed within first portion 306B. For example, second portion 306B may extend proximally within first portion 306A by a first length. In such a way, second portion 306B may be inverted within first portion to form a cavity 332. Cavity 332 may be configured such that a material 336 is disposed within cavity 332 of second portion 306B. An opening of cavity 332 may face distally.

First portion 306A and second portion 306B may be comprised of one or more flexible materials. For example, each of first portion 306A and second portion 306B may be comprised of rubber, latex, nylon, polyurethane, Pebax®, silicone, etc. First portion 306A and second portion 306B may form a continuous structure. For example, first portion 306A and second portion 306B may be joined at or adjacent to distal end of first portion 306A. In some embodiments, first portion 306A and second portion 306B may be the same material with the same characteristics (e.g., durometer, color, dimensions, etc.).

Alternatively, first portion 306A and second portion 306B may be comprised of the same material with different characteristics. For example, first portion 306A may be comprised of a clear or transparent first material and have a first durometer. Second portion 306B may be comprised of the same material, but have a lesser durometer and may be colored. In further alternative configurations, first portion 306A and second portion 306B may be comprised of different materials. Many other configurations of first portion 306A and second portion 306B are contemplated.

In the first configuration, a distal end of first portion 306A may be shaped, for example, to form a valve 324. For example, a wall forming first portion 306A may be formed towards a central axis A. Central axis A may extend longitudinally through distal portion 306, for example, from a proximal end to a distal end. Valve 324 may be shaped to permit material 336 to flow in a distal direction and prevent material flow in a proximal direction. In such a way, valve 324 may be a one-way valve.

In transitioning from the first configuration to the second configuration, material 336 may be delivered. To deliver material 336 from cavity 332, a fluid (e.g., a gas or a liquid) may be injected into first portion 306A. The injected fluid fills first portion 306A such that, as first portion 306A fills with the injected fluid, second portion 306B is forced distally. For example, second portion 306B may be forced out of first portion 306A such that second portion 306B extends distally from valve 324. In such a way, portion 306B and its cavity 332 are turned inside out to release material 336 through valve 324. For example, a length of distal portion 306 may increase in the second configuration.

A variety of materials may be delivered with distal portion 306. For example, material 336 may be a liquid, a powder, a gel, etc. Material 336 may additionally or alternatively include any compound or agent used for a diagnosis, a treatment, and/or palliation of a patient's disease or injury. For example, material 336 may include one or more hemostatic material(s) or agent(s).

In some embodiments, material 336 may be a patch. The patch may be pliable and bendable. For example, the patch may be formed into a planar film, sheet, or disc capable of being folded, crimped, or otherwise manipulated into a temporary non-planar configuration within cavity 332. Accordingly, in the first configuration, the patch may be folded, curved, or collapsed within cavity 332. As the patch is delivered in the second configuration, the patch may unfold or expand.

The patch may be a biodegradable and/or biocompatible patch of any suitable shape and any suitable dimension, e.g. based on the nature of the target tissue site. Moreover, the patch may be of any suitable color, including clear, and material, e.g., nettings, cloths, polysaccharides (chitosan, cellulose, starch, alginates, etc.) that may be further modified with synthetic biocompatible materials (pHEMA, PGA, PLA, PCA, PEG, etc.). Additionally, for example, the patch may be comprised of polypropylene, polyester, ePTFE, and/or silicone. Alternatively or additionally, the patch may comprise one or more hemostatic material(s) or agent(s). In some configurations, the hemostatic material(s) or agent(s) may be applied to the patch, for example, after the patch is delivered.

The patch may be substantially dry, for example, when the patch is disposed within cavity 332 and during delivery to the treatment site. For example, valve 324 may form a seal, or a liquid-tight barrier, to prevent bodily fluids from contacting the patch before delivery of deployment. The patch may then be moistened during or after delivery from distal portion 306. For example, the patch may be exposed to water or another fluid through contact with bodily fluids or the humidity inside the body of the subject. Alternatively, a fluid may be delivered to the treatment site. In such a way, as the patch is exposed to moisture, the patch may absorb the fluid, causing the patch to swell or expand. For example, when dry, the patch may be substantially paper-like, and, when wet, the patch may take on a more jelly-like consistency. Moistening the patch may assist in its application to the target tissue.

Distal portion 306 may be used similarly to distal portion 106 of FIG. 1. For example, distal portion 306 may be inserted into the subject, for example, though a natural orifice or an incision. In some examples, distal portion 306 may be inserted into the patient using an endoscope (not shown), for example, via a working channel of the endoscope. Once in the desired position, material 336 may be delivered. Material 336 may be delivered, for example, by injecting a fluid into first portion 306A such that second portion 306B is forced distally. Before, during, or after at least a portion of material 336 is delivered, the user(s) may reposition distal portion 306 within the subject. Once the desired volume of material 336 is delivered, the user(s) may remove distal portion 306 from the subject.

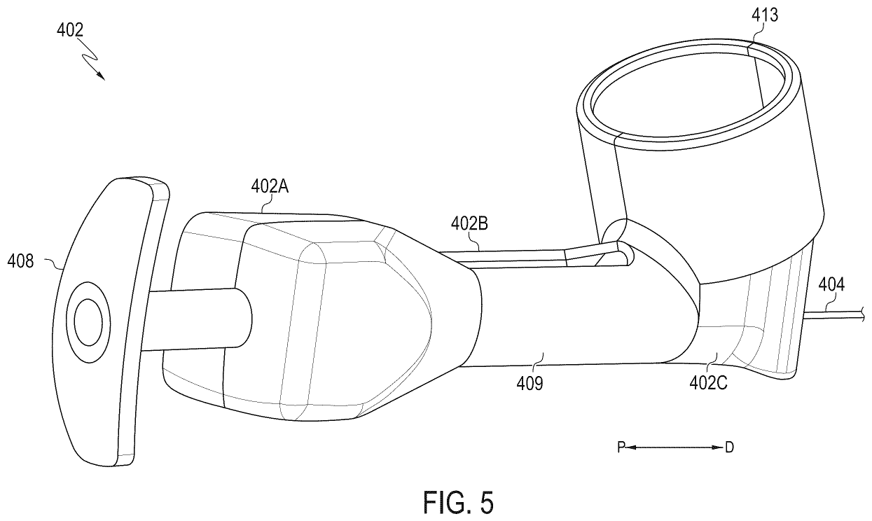

FIG. 5 illustrates a perspective view of an alternative handle 402. Handle 402 may be used with conduit 104, distal portion 106, and/or distal portion 306, described above. Handle 402 may include a proximal portion 402A, an intermediate portion 402B, and a distal portion 402C. Proximal portion 402A may include first movable body 408. Intermediate handle portion 402B and/or distal handle portion 402C may include a stationary body 409. Although not shown, stationary body 409 may include one or more ridges, grooves, or indentation, for example, to facilitate a user's grip on handle 402.

First movable body 408 may be a knob, a lever, a button, etc. First movable body 408 may be configured such that movement of first movable body 408 in a first direction (e.g., distally), delivers a fluid (e.g., a gas or a liquid) from handle 402, through conduit 404, and into a distal portion (e.g., distal portion 106 or distal portion 306, discussed above).

In some configurations, distal portion 402C of handle 402 may include at least one indicator 413. The at least one indicator 413 may be a gauge, an instrument, a device, etc., for example, configured to measure one or more aspects of the medical device. For example, indicator 413 may be configured to measure and/or display a volume, a pressure, a temperature, etc. of distal portion 106 shown in FIG. 1, 2A, or 2B or distal portion 306 shown in FIGS. 4A and 4B. Indicator 413 may include, for example, an analog display and/or a digital display.

Conduit 404 may be fixedly or removably coupled to a distal end of handle 402, for example, on a distal face of distal portion 402C. Conduit 404 may have any or all of the characteristics of conduit 104, discussed above with respect to FIG. 1. For example, conduit 404 may be formed by a tube, a sheath, a cannula, etc. Although not shown, a lumen may extend longitudinally though conduit 404, for example, from a proximal end to a distal end of conduit 404.

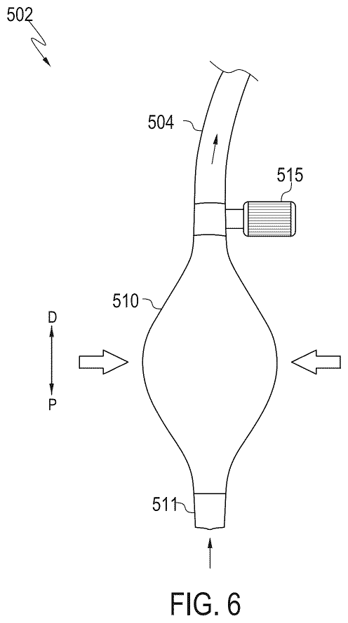

FIG. 6 illustrates a side view of a further alternative handle 502. Handle 502 may be used with conduit 104, distal portion 106, and/or distal portion 306, described above. Handle 502 may include an inflation bulb 510. Inflation bulb 510 may be coupled to a proximal end of a conduit 504. Inflation bulb 510 may be formed similar to an inflation bulb of a sphygmomanometer. For example, inflation bulb 510 may be compressed or squeezed one or more times to deliver a fluid (e.g., gas or liquid) to a distal portion (e.g., distal portion 106 and/or distal portion 306) of the medical device.

A proximal end of inflation bulb 510 may include a first valve 511. First valve 511 may permit fluid flow into inflation bulb 510, for example, to expand inflation bulb 510 after inflation bulb 510 has been compressed. For example, as inflation bulb 510 is compressed, first valve 511 is closed such that fluid only flows distally. As inflation bulb 510 is released and expands, first valve 511 opens to permit fluid flow into inflation bulb 510.

A distal end of inflation bulb 510 may include a second valve 515. Second valve 515 may be a deflation valve or a pressure release valve. For example, second valve 515 may be used to control fluid flow from inflation bulb 510. In some embodiments, second valve 515 may be used, for example, to release a pressure of the distal portion (not shown).

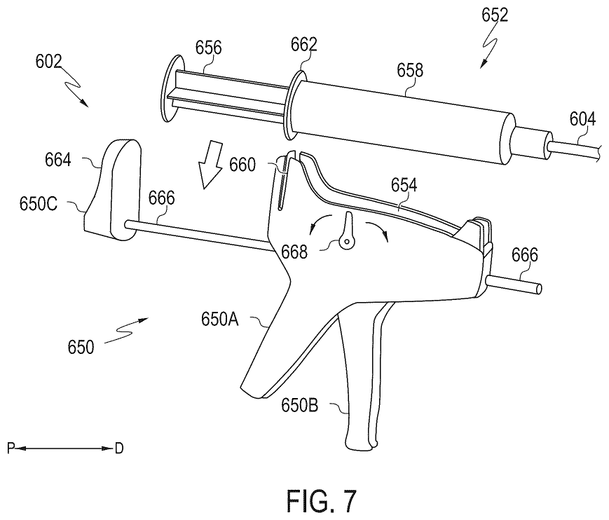

FIG. 7 illustrates a perspective view of a further alternative handle 602. Handle 602 may be used with conduit 104, distal portion 106, and/or distal portion 306, described above. Handle 602 may be comprised of two components. For example, handle 602 may include a grip 650 and a fluid container 652.

Fluid container 652 may be formed similar to a syringe. For example, fluid container 652 may include a plunger 656 movably disposed within a barrel 658. A fluid (e.g., a gas or a liquid) may be contained within barrel 658, for example, distal to plunger 656. A conduit 604 may be fixedly or removably coupled to a distal end of fluid container 652. In such a way, conduit 604 may be fluidly connected to fluid container 652. For example, as plunger 656 moves distally within barrel 658, the fluid contained within barrel 658 is forced distally into conduit 604. The fluid may thus be delivered to the distal portion coupled to conduit 604.

Conduit 604 may have any or all of the characteristics of conduit 104, discussed above with respect to FIG. 1. For example, conduit 604 may be formed by a tube, a sheath, a cannula, etc. Although not shown, a lumen may extend longitudinally though conduit 604, for example, from a proximal end to a distal end of conduit 604. In such a way, conduit 604 may permit fluid flow from handle 602 to a distal portion (e.g., distal portion 106 or distal portion 306).

Fluid container 652 may be used with grip 650. Grip 650 may be formed similar to a syringe gun or a dispending gun. For example, grip 650 is configured to receive fluid container 652. Grip 650 includes a stationary portion 650A and a first movable portion 650B. Fluid container may be seated within a groove 654 on stationary portion 650A of grip 650. Additionally, a slot 660 on stationary portion 650A may be configured to receive a flange 662 of fluid container 652. In such a way, barrel 658 may be fixed within grip 650.

With fluid container 652 seated in grip 650, first movable portion 650B may be moved, for example, to move a second movable portion 650C of grip 650 relative to stationary portion 650A. First movable portion 650B may be formed similar to a trigger. For example, first movable portion 650B may be pulled proximally relative to stationary portion 650A, thereby actuating first movable portion 650B. As first movable portion 650B is pulled proximally, second movable portion 650C is forced distally relative to stationary portion 650A. Second movable portion 650C may only move, for example, as first movable portion 650B is pulled proximally. In such a way, as first movable portion 650B is released and moves distally relative to stationary portion 650A, second movable portion 650C does not move. Accordingly, first movable portion 650B may be pulled proximally and released one or more times, for example, to move second movable portion 650C distally within stationary portion 650A.

Second movable portion 650C may include a pusher 664 and a rod 666 extending through stationary portion 650A. Pusher 664 may be configured to abut or touch a proximal face of plunger 656. Although not shown, rod 666 may include a plurality of teeth. The plurality of teeth may be configured to interact with first movable portion 650B, similar to a ratcheting rod. In such a way, as first movable portion 650B is actuated one or more times, second movable portion 650C is forced distally such that pusher 664 is forced against plunger 656. Accordingly, plunger 656 is forced distally within barrel 658 such that the fluid contained within barrel 658 is forced distally into conduit 604 and eventually into the distal portion coupled to conduit 604.

In some embodiments, stationary portion 650A may include an actuator 668 (e.g., a knob, a lever, a button, etc.) configured to change the direction of the movement of second movable portion 650C. For example, actuator 668 may be in a first position such that actuation of first movable portion 650B results in distal movement of second movable portion 650C. Alternatively, actuator 668 may be in a second position such that actuation of first movable portion 650B results in proximal movement of second movable portion 650C relative to stationary portion 650A. In such a way, the direction of fluid flow from fluid container 652 may be controlled.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

We claim:

1. A medical device comprising:
a handle having a fluid source;
a conduit coupled to the handle; and
a distal portion coupled to a distal end of the conduit, wherein the distal portion comprises:
an outer portion defining a lumen; and
an inner portion, wherein the inner portion defines a cavity having a distal opening, and wherein the cavity is configured to contain a material,
wherein, in a first configuration of the distal portion, the material is disposed within the inner portion and the inner portion is disposed within the lumen of the outer portion, and
wherein delivery of a fluid from the fluid source into the lumen of the outer portion transitions the distal portion from the first configuration to a second configuration and causes the inner portion to move distally relative to the outer portion and deliver at least a portion of the material.

2. The medical device of claim 1, wherein the fluid is a gas.

3. The medical device of claim 1, wherein the handle includes a first arm and a second arm, wherein the fluid source is a sac disposed between the first arm and the second arm,
wherein, upon movement of the first arm towards the second arm, the sac is compressed.

4. The medical device of claim 3, wherein the sac is corrugated.

5. The medical device of claim 1, wherein the distal portion is detachable from the conduit.

6. The medical device of claim 1, wherein:
in the first configuration, the inner portion has a first length;
in the second configuration, the inner portion has a second length; and
the first length is greater than the second length.

7. The medical device of claim 1, wherein the conduit is flexible, and wherein the distal portion is rigid or semi-rigid.

8. The medical device of claim 1, wherein the inner portion is a bladder.

9. The medical device of claim 8, wherein a color of the bladder indicates a volume of the bladder.

10. The medical device of claim 1, wherein the outer portion includes a distal valve through which the material is delivered.

11. The medical device of claim 10, wherein the distal valve is a one-way valve.

12. The medical device of claim 1, wherein the inner portion and the outer portion form one continuous structure.

13. The medical device of claim 12, wherein, in the first configuration, the inner portion is disposed within the outer portion, and wherein, in the second configuration, the inner portion extends distally relative to the outer portion.

14. The medical device of claim 13, wherein, in the second configuration, the inner portion is turned inside out to release the material.

15. The medical device of claim 1, wherein the material is a patch.

16. A distal end portion of a medical device, comprising:
an outer portion defining a lumen; and
an inner portion defining a cavity configured to contain a material;
wherein delivery of a fluid into the lumen of the outer portion transitions the distal portion from a first configuration to a second configuration and causes the inner portion to move distally relative to the outer portion, and wherein, in the second configuration, the inner portion is turned inside out to release at least a portion of the material.

17. The medical device of claim 16, wherein the material is a patch.

18. The medical device of claim 16, wherein the inner portion and the outer portion form one continuous structure.

19. The medical device of claim 16, wherein the distal end portion further includes a fitting on a proximal end of the distal end portion, wherein the fitting is configured to removably couple the distal end portion to a conduit.

20. A method of delivering a material, comprising:
inserting a distal end portion of a medical device into a subject, the distal end portion comprising:
an outer portion defining a lumen; and
an inner portion defining a cavity configured to contain a material,
wherein, during insertion of the distal end portion of the medical device into the subject, the inner portion is disposed within the lumen of the outer portion; and
delivering a fluid to the lumen of the outer portion, wherein, upon delivery of the fluid to the lumen of the outer portion, the inner portion moves distally relative to the outer portion to deliver at least a portion of the material.
